(19) United States
(12) Patent Application Publication
(10) Pub. No.: US 2003/0104421 A1
(43) Pub. Date: Jun. 5, 2003

(54) Methods and compositions for nucleic acid amplification

(76) Inventors: Christopher M. Colangelo, Old Lyme, CT (US); Jan Fredik Simons, New Haven, CT (US); Kendra Swirsding, Middletown, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/140,853

(22) Filed: May 7, 2002

Related U.S. Application Data
(60) Provisional application No. 60/289,331, filed on May 7, 2001.

(51) Int. Cl. C12Q 1/68; C07H 21/04

(52) U.S. Cl. 435/6; 536/24.3

(57) ABSTRACT

A plurality of oligonucleotides are provided having use in the amplification of target nucleic acid sequences. The oligonucleotides are used in simultaneous amplification of a multiplicity of nucleic acid sequences, providing a more balanced amplification of all target sequences. The oligonucleotides also provides a reaction system for the amplification of nucleic acid targets, and provide a method for selectively detecting one or more target nucleic acid sequences in a population of nucleic acid molecules, as well as a method for detecting a single nucleotide polymorphism in a population of nucleic acid molecules.

### RELATED APPLICATIONS

This application claims priority from U.S. provisional patent application serial no.: 60/289,331, filed May 7, 2001, the entirety of which is hereby incorporated herein by reference.

### FIELD OF THE INVENTION

This invention pertains to universal oligonucleotides having use in amplification of nucleic acid sequences, specifically to oligonucleotides that allow the simultaneous amplification of a multiplicity of nucleic acid sequences.

### BACKGROUND OF THE INVENTION

Polymerase chain reaction (PCR) is a method whereby virtually any DNA sequence can be selectively amplified. The method involves using paired sets of oligonucleotides of predetermined sequence that hybridize to opposite strands of DNA and define the limits of the sequence to be amplified. The oligonucleotides prime multiple sequential rounds of DNA synthesis catalyzed by a thermostable DNA polymerase. Each round of synthesis is typically separated by a melting and re-annealing step, allowing a given DNA sequence to be amplified several hundred-fold in less than an hour (Saiki et al., Science 239:487, 1988).

With the rapid advances in mammalian molecular genetics, an ever increasing number of disease genes have been identified. Accordingly, PCR has gained widespread use for the diagnosis of inherited disorders and the susceptibility to disease. Typically, the region of interest is amplified from either genomic DNA or from a source of specific cDNA encoding the cognate gene product. Mutations or polymorphisms are then identified by subjecting the amplified DNA to analytical techniques such as DNA sequencing, hybridization with allele-specific oligonucleotides (ASOs), oligonucleotide ligation, restriction endonuclease cleavage or single-strand conformational polymorphism (SSCP) analysis.

For the analysis of small genes and transcripts, or genes where the mutant allele or polymorphism is well characterized, amplification of a single defined region of the target nucleic acid is sometimes sufficient. When analyzing large genes and transcripts or undefined genes, however, multiple individual amplification reactions are often required to identify critical base changes or deletions. Thus, to streamline the analysis of large complex genes, multiplex amplification (i.e., the simultaneous amplification of different target sequences in a single reaction) has been utilized.

The results obtained with multiplex amplification are, however, frequently complicated by artifacts of the amplification procedure. These include “false-negative” results due to reaction failure and “false-positive” results such as the amplification of spurious products, which may be caused by annealing of the primers to sequences which are related to, but distinct from, the true recognition sequences.

For use in multiplex amplification, a oligonucleotide should be designed so that its predicted hybridization kinetics are similar to those of the other primers used in the same multiplex reaction. While the annealing temperatures and primer concentrations may be calculated to some degree, conditions generally have to be empirically determined for each multiplex reaction. Since the possibility of non-specific priming increases with each additional primer pair, conditions must be modified as necessary as individual primer sets are added. Moreover, artifacts that result from competition for resources (e.g., depletion of primers) are augmented in multiplex amplification, since differences in the yields of unequally amplified fragments are enhanced with each cycle.

Weighardt et al. (PCR Meth.App. 3:77, 1993) describe the use of 5′-tailed oligonucleotides for PCR. However, a key feature of this amplification method involves separate annealing and primer extension reactions for each primer, which is not practical in a multiplex context. Therefore, complete optimization of the reaction conditions for multiplex amplification can become labor intensive and time consuming. Since different multiplex amplifications may each have unique reaction conditions, development of new diagnostic tests can become very costly.

### SUMMARY OF THE INVENTION

The present invention allows multiplexing of nucleic acid amplification, such that equal amplification of multiple products can be obtained from the same reaction using the same template. The methods and compositions of the present invention can be applied to diverse applications where multiplex amplification of nucleic acids with close preservation of original template ratios is desirable, for example, the diagnosis of genetic and infectious diseases, gender determination, genetic linkage analysis, and forensic studies, gene expression analysis by determining the relative abundance of specific cDNAs in an mRNA-derived cDNA pool, and the like.

In one embodiment, the invention provides a plurality of oligonucleotides comprising a first oligonucleotide comprising an A subsequence and a B subsequence. The A subsequence is provided at the 5′ terminus of the first oligonucleotide, which does not hybridize to the target nucleic acid sequence. The B subsequence is provided at the 3′ terminus of the first oligonucleotide, and hybridizes to a target nucleic acid sequence. The plurality of oligonucleotides further comprises a second oligonucleotide having an A subsequence at its 3′ terminus which does not hybridize to the target nucleic acid sequence. In another embodiment, the invention provides a first reverse oligonucleotide comprising an A subsequence and a B′ subsequence, wherein the B′ subsequence is provided at the 3′ terminus of the first reverse oligonucleotide, and wherein the B′ subsequence hybridizes to a target nucleic acid sequence and can be extended to form an extension product comprising a sequence complementary to said B subsequence, and the A subsequence does not hybridize to said target nucleic acid sequence. In one aspect, the second oligonucleotide is present at a higher concentration than the first oligonucleotide. In another aspect, the second oligonucleotide is present at a higher concentration than the first reverse oligonucleotide. In yet another aspect, for example, the second oligonucleotide is present at about a 2, 5, 10, 15, 20, or 50-fold higher molar concentration than the first or the first reverse oligonucleotide. In one aspect the first oligonucleotide or the first reverse oligonucleotide is about 20 to about 80 nucleotides in length, and the second oligonucleotide is about 10 to about 70 nucleotides in length.

In one embodiment, the invention further comprises a third oligonucleotide comprising the A subsequence at its 5′ terminus and a C subsequence at its 3′ terminus, wherein the C subsequence hybridizes to a target nucleic acid sequence and the A subsequence does not hybridize to the target nucleic acid sequence. In one aspect the invention further comprises a third reverse oligonucleotide comprising the A subsequence and a C′ subsequence, wherein the C′ subsequence is provided at the 3′ terminus of said oligonucleotide, and wherein the C′ subsequence hybridizes to a target nucleic acid sequence and can be extended to form an extension product comprising a sequence complementary to said C subsequence, and the A subsequence does not hybridize to the target nucleic acid sequence.

In one embodiment, the invention further comprises a fourth oligonucleotide comprising the A subsequence at its 5′ terminus and a D subsequence at its 3′ terminus, wherein the D subsequence hybridizes to a target nucleic acid sequence and the A subsequence does not hybridize to the target nucleic acid sequence. In one aspect the invention further comprises a fourth reverse oligonucleotide comprising the A subsequence and a D′ subsequence, wherein the D′ subsequence is provided at the 3′ terminus of said oligonucleotide, and wherein the D′ subsequence hybridizes to a target nucleic acid sequence and can be extended to form an extension product comprising a sequence complementary to said D subsequence, and the A subsequence does not hybridize to the target nucleic acid sequence.

In one embodiment, the invention further comprises a fifth oligonucleotide comprising the A subsequence at its 5′ terminus and an E subsequence at its 3′ terminus, wherein the E subsequence hybridizes to a target nucleic acid sequence and the A subsequence does not hybridize to the target nucleic acid sequence. In one aspect the invention further comprises a fifth reverse oligonucleotide comprising the A subsequence and a E′ subsequence, wherein the E′ subsequence is provided at the 3′ terminus of said oligonucleotide, and wherein the E′ subsequence hybridizes to a target nucleic acid sequence and can be extended to form an extension product comprising a sequence complementary to said E subsequence, and the A subsequence does not hybridize to the target nucleic acid sequence.

In yet another embodiment, the invention comprises a reaction system for selectively detecting one or more target nucleic acid sequences in a population of nucleic acid molecules, the reaction system comprising a population of starting nucleic acid molecules known to or suspected of containing at least one target nucleic acid sequence. In this embodiment, the reaction system comprises the plurality of oligonucleotides, i.e., the first oligonucleotide, the first reverse oligonucleotide, and the second oligonucleotide. In one aspect the reaction system additionally comprises the third and third reverse oligonucleotides. In another aspect, the reaction system additionally comprises the fourth and fourth reverse oligonucleotides, and optionally the fifth and fifth reverse oligonucleotides, or additional target specific oligonucleotides to provide multiplex amplification of target nucleic acid sequences. In one aspect the reaction system comprises a polymerase. In another aspect the polymerase is a thermostable nucleic acid polymerase. In one aspect the thermostable nucleic acid polymerase is, for example, one or more of the DNA polymerases from Bacillus stearothermophilus, Thermus aquaticus, Pyrococcus furiosis, Thermococcus litoralis, and Thermus thermophilus, bacteriophage T4 and T7, the E. coli DNA polymerase I Klenow fragment, the reverse transcriptase from the Avian Myeloblastosis Virus, the reverse transcriptase from the Moloney Murine Leukemia Virus, and the reverse transcriptase from the Human Immunodeficiency Virus-I or combinations of these enzymes.

In still another embodiment, the invention provides a kit for selectively detecting one or more target nucleic acid sequences in a population of nucleic acid molecules, the kit comprising the plurality of oligonucleotides described above, and further comprising an instruction set for using the kit. In one aspect the kit further comprises a polymerase. In another aspect the kit comprises a thermostable nucleic acid polymerase. In yet another aspect the kit comprises one or more of the DNA polymerases from Bacillus stearothermophilus, Thermus aquaticus, Pyrococcus furiosis, Thermococcus litoralis, and Thermus thermophilus, bacteriophage T4 and T7, the E. coli DNA polymerase I Klenow fragment, the reverse transcriptase from the Avian Myeloblastosis Virus, the reverse transcriptase from the Moloney Murine Leukemia Virus, and the reverse transcriptase from the Human Immunodeficiency Virus-I or combinations of these enzymes.

In one embodiment, the invention provides a method for selectively detecting one or more target nucleic acid sequences in a population of nucleic acid molecules, the method comprising contacting a population of starting nucleic acid molecules known to or suspected of containing at least one target nucleic acid sequence with an effective amount of a first oligonucleotide and an effective amount of a first reverse oligonucleotide to form a primed first oligonucleotide complex and a primed first reverse oligonucleotide complex, where the first oligonucleotide has an A subsequence and a B subsequence, wherein said B subsequence is provided at the 3′ terminus of said oligonucleotide, and wherein the B subsequence hybridizes to the target nucleic acid sequence and the A subsequence does not hybridize to the target nucleic acid sequence, thereby forming an annealed first oligonucleotide-target nucleotide complex; said first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to a target nucleic acid sequence and can be extended to form an extension product comprising a sequence complementary to said B subsequence, and the A subsequence does not hybridize to the target nucleic acid sequence, thereby forming an annealed first reverse oligonucleotide-target nucleotide complex; extending said annealed first oligonucleotide complex and annealed first reverse oligonucleotide complex with a polymerase to form a first extended oligonucleotide sequence and first reverse extended oligonucleotide sequence; combining said first extended oligonucleotide sequence and first reverse extended oligonucleotide sequence with an effective amount of a second oligonucleotide to form annealed second oligonucleotide complex, wherein said second oligonucleotide has a 5′ terminus and a 3′ terminus, the second nucleotide comprising the A subsequence at its 3′ terminus; extending the annealed second oligonucleotide-target nucleotide complex and annealed first reverse oligonucleotide-target nucleotide complex, thereby forming extended second oligonucleotide sequences; and detecting said extended second oligonucleotide sequences, thereby selectively amplifying one or more target nucleic acid sequences in a population of nucleic acid molecules. In one aspect, the method further comprises contacting said starting population of nucleic acid molecules with said second oligonucleotide. In another aspect, the second oligonucleotide is present at a higher concentration than said first oligonucleotide. In yet another aspect, extending the annealed second oligonucleotide-target nucleotide complex and annealed first reverse oligonucleotide-target nucleotide complex, thereby forming extended second oligonucleotide sequences is effectuated with a polymerase.

In yet another embodiment, the invention provides a method for detecting a single nucleotide polymorphism in a population of nucleic acid molecules, the method comprising contacting a population of starting nucleic acid molecules known to or suspected of containing at least one polymorphic nucleic acid sequence with an effective amount of a first oligonucleotide and an effective amount of a first reverse oligonucleotide to form a primed first oligonucleotide complex and a primed first reverse oligonucleotide complex, the first oligonucleotide having a 5′ terminus and a 3′ terminus and comprising an A subsequence and a B subsequence, wherein the B subsequence is provided at the 3′ terminus of the oligonucleotide, and wherein the B subsequence hybridizes to the polymorphic nucleic acid sequence and the A subsequence does not hybridize to the polymorphic nucleic acid sequence, thereby forming an annealed first oligonucleotide-polymorphic nucleotide complex; the first reverse oligonucleotide having a 5′ terminus and a 3′ terminus and comprising an A subsequence and a B′ subsequence, wherein said B′ subsequence is provided at the 3′ terminus of the oligonucleotide, and wherein the B′ subsequence hybridizes to a polymorphic nucleic acid sequence and can be extended to form an extension product comprising a sequence complementary to the B subsequence, and the A subsequence does not hybridize to the polymorphic nucleic acid sequence, thereby forming an annealed first reverse oligonucleotide-target nucleotide complex; extending the annealed first oligonucleotide complex and annealed first reverse oligonucleotide complex with a polymerase to form a first extended oligonucleotide sequence and first reverse extended oligonucleotide sequence; combining the first extended oligonucleotide sequence and first reverse extended oligonucleotide sequence with an effective amount of a second oligonucleotide to form annealed second oligonucleotide complex, wherein the second oligonucleotide has a 5′ terminus and a 3′ terminus, the second nucleotide comprising said A subsequence at its 3′ terminus; extending the annealed second oligonucleotide-polymorphic nucleotide complex and annealed first reverse oligonucleotide-target nucleotide complex with a polymerase, thereby forming extended second oligonucleotide sequences; and detecting the extended second oligonucleotide sequences, thereby identifying a single nucleotide polymorphic sequence in a population of nucleic acid molecules. In one aspect, the method further comprises contacting the starting population of nucleic acid molecules with a second oligonucleotide. In one aspect, the second oligonucleotide is present at a higher concentration than said first oligonucleotide, for example, from about a 2 to about a 50 molar excess.

### BRIEF DESCRIPTION OF THE DRAWINGS

FIG. 1 illustrates a multiplex polymerase chain reaction according to the present invention. Four single nucleotide polymorphisms (SNP's) are amplified in a single reaction.

FIG. 2 illustrates amplification of target sequences, comparing a standard PCR reaction with the multiplex PCR reaction. The reaction products are resolved by agarose gel electrophoresis.

FIG. 3 illustrates MegaBACE runs of the amplification products from standard or multiplex PCR reactions.

FIG. 4 illustrates PCR amplification reactions of a two-plexed sample, where the ratio between the target specific oligonucleotides was adjusted to equalize the resultant products. Panel A illustrates agarose gel electrophoresis of the reaction products from standard or two-plexed PCR reactions. Panel B illustrates MegaBACE traces of the resulting amplification products.

FIG. 5 illustrates the formation of primer dimers in an amplification reaction. Panel A illustrates an MegaBACE trace of a two-plexed amplification. Panel B illustrates the same two-plexed amplification, using a two-fold reduction in the concentration of the oligonucleotides. Panel C illustrates the two-plexed amplification using the reduced oligonucleotide concentrations shown in Panel B, and with the addition of chain terminating dideoxynucleotide triphosphates and a 94° C. denaturing step.

FIG. 6 illustrates MegaBACE traces of the resulting amplification products of polymorphic targets using various dye terminator combinations.

FIG. 7 illustrates traces of the amplification products before and following digestion of the unincorporated dye-conjugated free nucleotides with shrimp alkaline phosphatase (SAP).

### DETAILED DESCRIPTION OF THE INVENTION

The invention described herein provides compositions and methods for multiplex amplification of nucleic acid target sequences. A plurality of oligonucleotides is used, whereby oligonucleotides having target specific sequences, and capable of hybridizing thereto, are used with an oligonucleotide having a common sequence that does not hybridize to the target sequences. During the first round of amplification, the target specific oligonucleotides hybridize to the target sequences, and result in intermediate amplification products having the target sequences and the common sequence. The second oligonucleotide can hybridize to these intermediate amplification products, irrespective of their target sequences, thereby providing a more balanced multiplex amplification by providing simultaneous amplification of one or more intermediate amplification products having the nucleic acid target sequences.

The present invention offers many advantages over other multiplex amplification processes. Without being bound to theory, it is believed that the more balanced amplification seen with the invention results from the oligonucleotide having the common sequence hybridizing to all previous amplification products. This oligonucleotide, being universal for all intermediate amplification products, is used in excess of the target specific oligonucleotides.

The examples provided below demonstrate that multiple target sequences can be co-amplified under identical reaction conditions and cycling parameters with very little optimization of conditions. Using the compositions and methods of the present invention, highly specific and efficient amplification of target sequences can be easily and reproducibly achieved by simple adjustment of the individual oligonucleotide concentrations, with no additional modification of either the reaction components or conditions.

Definitions

As used herein “amplification” of a target sequence denotes an increase in the concentration of a particular nucleic acid moiety comprising the target sequence, from a nucleic acid template comprising a plurality of sequences. An “amplicon” or “intermediate amplification product” is the nucleic acid moiety comprising the target sequence, amplified by an extension reaction, i.e., polymerase chain reaction, primer extension, or rolling circle replication.

As used herein “multiplex amplification” refers to the simultaneous amplification of one or more target sequences in a single mixture. For example, a two-plex amplification refers to the simultaneous amplification in a single reaction mixture, of two amplicons comprising different target sequences.

As used herein “Tₘ” refers to the melting temperature of a nucleic acid i.e., the temperature at which one-half of the nucleic acid exists in the form of a duplex and one-half of the nucleic acid exists in a single stranded form. The Tₘ is a function of several variables including the length of the nucleic acid, its chemical makeup, and the ionic strength of the solvent in which the nucleic acid is mixed. Calculation of Tₘ for a nucleic acid is well known in the art, for example using the nearest-neighbor thermodynamic values of, for DNA, Breslauer et al., Proc. Natl. Acad. Sci. USA 83:3746-3750, 1986, and for RNA, Freier et al., Proc. Natl. Acad. Sci. USA 83:9373-9377, 1986, each incorporated by reference in their entirety.

As used herein “ΔG” refers to the free energy for the nucleic acid. The free energy is a measure of stability, for example, the greater the negative value, the more stable the duplex formed by the nucleic acid. The free energy is calculated by the formula: G=H−TS; where H is the enthalpy, S is the entropy and T is the temperature. Calculation of ΔG is known to those skilled in the art, and is described for nucleic acids in the Breslauer et al., 1986 and Freier et al., 1986 references cited above.

Oligonucleotide Design

Multiplex amplification according to present invention utilizes a plurality of oligonucleotides. The design of such oligonucleotides for multiplex amplification are well known in the art, and the design and use of such oligonucleotides in, for example, PCR are discussed in U.S. Pat. Nos. 6,207,372, 5,882,856, 5,736,365, 5,624,825, and 5,104,792, each incorporated herein by reference in their entirety. For use in a given multiplex amplification reaction, oligonucleotide sequences are typically analyzed as a group to evaluate the potential for fortuitous dimer formation between different oligonucleotides. This evaluation may be achieved using commercially available computer programs for sequence analysis, such as Gene Runner, Hastings Software Inc. Other variables, such as the preferred concentrations of Mg^{2+,} dNTPs, polymerase, and primers, are optimized using methods well-known in the art (Edwards et al., PCR Meth. App. 3:565,1994, incorporated herein by reference).

A multiplex amplification according to the present invention utilizes a first oligonucleotide having a target specific subsequence B, a first reverse oligonucleotide having a target specific subsequence B′, and a second oligonucleotide having a common or universal subsequence A that is not target specific. The first, first reverse, and second oligonucleotides are thus used in an amplification reaction, to amplify a target sequence having the subsequences B and B′, thereby generating amplicons having the subsequences A, B, and B′.

The first oligonucleotide is designed such that it has a 5′ terminus and a 3′ terminus and comprises an A subsequence and a B subsequence. The termini may be discontinuous, for example, a linear oligonucleotide, or the termini may be contiguous, for example, in a rolling circle replication. The A subsequence does not hybridize to a target nucleic acid sequence and is provided at the 5′ terminus of said first oligonucleotide. An A subsequence is from about 8 to about 40 bases in length, and more preferably from about 12 to about 25 bases in length. The B subsequence hybridizes to a target nucleic acid sequence and is provided at the 3′ terminus of said first oligonucleotide. A B subsequence is from about 8 to about 40 bases in length, and more preferably from about 12 to about 25 bases in length. In an amplification, the first oligonucleotide is extended to form an extension product comprising a sequence complementary to the B subsequence.

The first reverse oligonucleotide has a 5′ terminus and a 3′ terminus and comprises an A subsequence and a B′ subsequence. The A subsequence does not hybridize to the target nucleic acid sequence, and is provided at the 5′ terminus of said first reverse oligonucleotide. An A subsequence is from about 8 to about 40 bases in length, and more preferably from about 12 to about 25 bases in length. The B′ subsequence hybridizes to a target nucleic acid, and is provided at the 3′ terminus of said first reverse oligonucleotide sequence. A B′ subsequence is from about 8 to about 40 bases in length, and more preferably from about 12 to about 25 bases in length. In an amplification, the first reverse oligonucleotide is extended to form an extension product comprising a sequence complementary to said B′ subsequence.

A second oligonucleotide has a 5′ terminus and a 3′ terminus, comprising said A subsequence at its 3′ terminus or its 5′ terminus. The A subsequence does not hybridize to the target nucleic acid sequences B or B′, but does hybridize to the A subsequences of the first and first reverse oligonucleotides. The second oligonucleotide also hybridizes with high stringency to the A subsequences of amplicons, or extension products formed by extension of the first and first reverse oligonucleotides. An A subsequence of a second oligonucleotide is from about 8 to about 40 bases in length, and more preferably from about 12 to about 25 bases in length. The second oligonucleotide is present at a higher concentration than the first and first reverse oligonucleotides, for example, the second oligonucleotide is present at about a 2, 5, 10, 15, 20, or 50-fold higher molar concentration than said first oligonucleotide.

In a multiplex amplification, where it is desirable to simultaneously amplify a second target sequence having subsequences C and C′, a third oligonucleotide and third reverse oligonucleotide are added to the amplification reaction mixture.

The third oligonucleotide is designed such that it has a 5′ terminus and a 3′ terminus and comprises an A subsequence and a C subsequence. The A subsequence does not hybridize to a target nucleic acid sequence and is provided at the 5′ terminus of said third oligonucleotide. An A subsequence is from about 8 to about 40 bases in length, and more preferably from about 12 to about 25 bases in length. The C subsequence hybridizes to a target nucleic acid sequence and is provided at the 3′ terminus of said third oligonucleotide. A C subsequence is from about 8 to about 40 bases in length, and more preferably from about 12 to about 25 bases in length. In an amplification, the third oligonucleotide is extended to form an extension product comprising a sequence complementary to said C subsequence.

A third reverse oligonucleotide has a 5′ terminus and a 3′ terminus and comprises an A subsequence and a C′ subsequence. The A subsequence does not hybridize to the target nucleic acid sequence, and is provided at the 5′ terminus of said third reverse oligonucleotide. An A subsequence is from about 8 to about 40 bases in length, and more preferably from about 12 to about 25 bases in length. The C′ subsequence hybridizes to a target nucleic acid, and is provided at the 3′ terminus of said third reverse oligonucleotide sequence. A C′ subsequence is from about 8 to about 40 bases in length, and more preferably from about 12 to about 25 bases in length. In an amplification, the third reverse oligonucleotide is extended to form an extension product comprising a sequence complementary to said B′ subsequence.

The second oligonucleotide hybridizes with high stringency to the A subsequences of the resultant amplicons, i.e., extension products formed by extension of the third and third reverse oligonucleotides, as well as the first and first reverse oligonucleotides, thereby functioning as universal oligonucleotides and providing simultaneous amplification of the first and second target sequences.

Where the invention is used in a multiplex amplification of a third target sequence, a target specific fourth oligonucleotide and a fourth reverse oligonucleotide are employed. These are designed as described above for the first and first reverse oligonucleotides, but comprise the third target specific subsequences D and D′ instead of the first target specific sequences B and B′. Similarly, multiplex amplification of a fourth target sequence can be obtained by using a fifth oligonucleotide and fifth reverse oligonucleotide having the fourth target specific subsequences E and E′. Thus a multiplex amplification of one or more target sequences is provided. One, two, three, four, five, six, seven, eight, nine, ten or more target sequences can be amplified, by using a plurality of oligonucleotides as described herein. In all cases, the second oligonucleotide having the A and A′ subsequences function as universal oligonucleotides for the multiplex amplification of extension products having different (B and B′, C and C′, D and D′ etc.) target sequences.

Nucleic Acid Templates

Any nucleic acid sample may be used as a template in practicing the present invention, including without limitation eukaryotic, prokaryotic and viral nucleic acids, such as polyA+RNA, mRNA, tRNA, aptamers, ribozymes, genomic DNA, cDNA, ssDNA, or chemically modified nucleic acids, for example tailed nucleic acids. These template nucleic acids may be from genomic or episomal sources, or from organelles such as mitochondria. The template nucleic acid can be constructed from any source of nucleic acid, e.g., any cell, tissue, or organism, and can be generated by any art-recognized method. Suitable methods include, e.g., sonication of genomic DNA and digestion with one or more restriction endonucleases (RE) to fragment a population of nuclei acid molecules, e.g., genomic DNA. Preferably, one or more of the restriction enzymes have distinct four-base recognition sequences. Examples of such enzymes include, e.g., Sau3A1, MspI, and TaqI. Preferably, the enzymes are used in conjunction with oligonucleotides having regions containing recognition sequences for the corresponding restriction enzymes. Oligonucleotides may contain additional sequences adjoining known restriction enzyme recognition sequences, thereby allowing for capture or annealing of specific restriction fragments of interest to the oligonucleotide. In a currently preferred embodiment, the target nucleic acid is a sample of genomic DNA isolated from a patient. This may be obtained from any cell source or body fluid by methods well known to those skilled in the art. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen and tissue exudates at the site of infection or inflammation. It will be understood that the particular method used to extract nucleic acids will depend on the nature of the source. The preferred amount of nucleic acid to be extracted for use in the present invention is at least 5 pg (corresponding to about 1 cell equivalent of a genome size of 4×10⁹ base pairs). However, amplification of target sequences can be performed with about femtogram quantities of nucleic acids, such as in a PCR amplification. One skilled in the art would understand which amplification method to use in view of the quantity and source of the nucleic acid template.

Multiplex Amplification Reaction Conditions

In practicing the present invention, a nucleic acid sample is contacted with oligonucleotides as described under conditions suitable for amplification of target sequences contained on the nucleic acid sample. Multiplex amplification reactions are carried out using manual or automatic methods. A number of in vitro nucleic acid amplification techniques have been described. These amplification methodologies may be differentiated into those methods: (i) which require temperature cycling—polymerase chain reaction (PCR) (see e.g., Saiki, et al., 1995. Science 230: 1350-1354), ligase chain reaction (see e.g., Barany, 1991. Proc. Natl. Acad. Sci. USA 88: 189-193; Barringer, et al., 1990. Gene 89: 117-122) and transcription-based amplification (see e.g., Kwoh, et al., 1989. Proc. Natl. Acad. Sci. USA 86: 1173-1177) and (ii) isothermal amplification systems—self-sustaining, sequence replication (see e.g., Guatelli, et al., 1990. Proc. Natl. Acad. Sci. USA 87: 1874-1878); the Qβ replicase system (see e.g., Lizardi, et al., 1988. BioTechnology 6: 1197-1202); strand displacement amplification Nucleic Acids Res. Apr. 11, 1992;20(7):1691-6.; and the methods described in PNAS Jan. 1, 1992;89(1):392-6; and NASBA J Virol Methods. 1991 Dec;35(3):273-86, each incorporated herein by reference in their entirety.

Isothermal amplification also includes rolling circle-based amplification (RCA). RCA is discussed in, e.g., Kool, U.S. Pat. No. 5,714,320, Lizardi, U.S. Pat. No. 5,854,033; Hatch, et al., 1999. Genet. Anal. Biomol. Engineer. 15: 35-40, and Rothberg et al, U.S. Pat. No. 6,274,320, each incorporated herein by reference in their entirety. The result of the RCA is a single DNA strand extended from the 3′ terminus of the first or first reverse oligonucleotide and including a concatamer containing multiple copies of the circular template annealed to the oligonucleotide. Typically, 10,000 or more copies of circular templates, each having a size of approximately 100 nucleotides size range, can be obtained with RCA.

Circular oligonucleotides which are generated during polymerase-mediated replication are dependent upon the relationship between the template and the site of replication initiation. In double-stranded templates, the critical features include whether the template is linear or circular in nature, and whether the site of initiation of replication (i.e., the replication “fork”) is engaged in synthesizing both strands of DNA or only one. In conventional double-stranded DNA replication, the replication fork is treated as the site at which the new strands of DNA are synthesized. However, in linear molecules (whether replicated unidirectionally or bidirectionally), the movement of the replication fork(s) generate a specific type of structural motif. If the template is circular, one possible spatial orientation of the replicating molecule takes the form of a theta structure. Alternatively, RCA can occur when the replication of the duplex molecule begins at the origin. Subsequently, a nick opens one of the strands, and the free 3′-terminal hydroxyl moiety generated by the nick is extended by the action of DNA polymerase. The newly synthesized strand eventually displaces the original parental DNA strand. This aforementioned type of replication is known as rolling-circle replication (RCR) because the point of replication may be envisaged as “rolling around” the circular template strand and, theoretically, it could continue to do so indefinitely. As it progresses, the replication fork extends the outer DNA strand beyond the previous partner. Additionally, because the newly synthesized DNA strand is covalently-bound to the original template, the displaced strand possesses the original genomic sequence (e.g., gene or other sequence of interest) at its 5′-terminus. In rolling-circle replication, the original genomic sequence is followed by any number of “replication units” complementary to the original template sequence, wherein each replication unit is synthesized by continuing revolutions of said original template sequence. Hence, each subsequent revolution displaces the DNA which is synthesized in the previous replication cycle.

In vivo, rolling-circle replication is utilized in several biological systems. For example, in certain bacteriophage, their genome consists of single-stranded, circular DNA. During replication, the circular DNA is initially converted to a duplex form, which is then replicated by the aforementioned rolling-circle replication mechanism. The displaced terminus generates a series of genomic units, which can be cleaved and inserted into the phage particles, or they can be utilized for further replication cycles by the phage. Additionally, the displaced single-strand of a rolling-circle can be converted to duplex DNA by synthesis of a complementary DNA strand. This synthesis can be used to generate the concatemeric duplex molecules required for the maturation of certain phage DNAs. For example, this provides the principle pathway by which .lambda. bacteriophage matures. Rolling-circle replication is also used in vivo to generate amplified rDNA in Xenopus oocytes, and this fact may help explain why the amplified rDNA is comprised of a large number of identical repeating units. In this case, a single genomic repeating unit is converted into a rolling-circle. The displaced terminus is then converted into duplex DNA which is subsequently cleaved from the circle so that the two termini can be ligated together so as to generate the amplified circle of rDNA. Through the use of the RCR reaction, a strand may be generated which represents many tandem copies of the complement to the circularized molecule. For example, RCR has recently been utilized to obtain an isothermal cascade amplification reaction of circularized padlock probes in vitro in order to detect single-copy genes in human genomic DNA samples (see Lizardi, et al., 1998. Nat. Genet. 19: 225-232). In addition, RCR has also been utilized to detect single DNA molecules in a solid phase-based assay, although difficulties arose when this technique was applied to in situ hybridization (see Lizardi, et al., 1998. Nat. Genet. 19: 225-232).

The development of a method of amplifying short DNA molecules which have immobilized to a solid support, termed rolling circle amplification (RCA) has been recently described in the literature (see e.g., Hatch, et al, 1999, incorporated herein by reference). Rolling circle amplification of DNA immobilized on solid surfaces and its application to multiplex mutation detection. Genet. Anal. Biomol. Engineer. 15: 35-40; Zhang, et al., 1998, incorporated herein by reference. Amplification of target-specific, ligation-dependent circular probe. Gene 211: 277-85; Baner, et al., 1998, incorporated herein by reference. Signal amplification of padlock probes by rolling circle replication. Nucl. Acids Res. 26: 5073-5078; Liu, et al., 1995. Rolling circle DNA synthesis: small circular oligonucleotides as efficient templates for DNA polymerase. J. Am. Chem. Soc. 118: 1587-1594; Fire and Xu, 1995. Rolling replication of short DNA circles. Proc. Natl. Acad. Sci. USA 92: 4641-4645; Nilsson, et al., 1994. Padlock probes: circularizing oligonucleotides for localized DNA detection. Science 265: 2085-2088), each incorporated herein by reference. RCA targets specific DNA sequences through hybridization and a DNA ligase reaction. The circular product is then subsequently used as a template in a rolling circle replication reaction.

Rolling-circle amplification (RCA) driven by DNA polymerase can replicate circularized oligonucleotide probes with either linear or geometric kinetics under isothermal conditions. In the presence of two primers (one hybridizing to the + strand, and the other, to the − strand of DNA), a complex pattern of DNA strand displacement ensues which possesses the ability to generate 10⁹ or more copies of each circle in a short period of time (i.e., less-than 90 minutes), enabling the detection of single-point mutations within the human genome. Using a single primer, RCA generates hundreds of randomly-linked copies of a covalently closed circle in several minutes. If solid support matrix-associated, the DNA product remains bound at the site of synthesis, where it may be labeled, condensed, and imaged as a point light source. For example, linear oligonucleotide probes, which can generate RCA signals, have been bound covalently onto a glass surface. The color of the signal generated by these probes indicates the allele status of the target, depending upon the outcome of specific, target-directed ligation events. As RCA permits millions of individual probe molecules to be counted and sorted, it is particularly amenable for the analysis of rare somatic mutations. RCA also shows promise for the detection of padlock probes bound to single-copy genes in cytological preparations. Other examples of isothermal amplification systems include, e.g., (i) self-sustaining, sequence replication (see e.g., Guatelli, et al., 1990. Proc. Natl. Acad. Sci. USA 87: 1874-1878), (ii) the Q.beta. replicase system (see e.g., Lizardi, et al., 1988. BioTechnology 6: 1197-1202), and (iii) nucleic acid sequence-based amplification (NASBA.TM.; see Kievits, et al., 1991. J. Virol. Methods 35: 273-286, each incorporated herein by reference).

One other method of amplifying a nucleic acid sequence involves polymerase chain reaction. Examples of such multiplex PCR amplifications can be found in U.S. Pat. No. 5,104,792 “Method for Amplifying Unknown Nucleic Acid Sequences”, U.S. Pat. No. 5,624,825 “Simultaneous Amplification of Multiple Targets”, U.S. Pat. No. 5,736,365 “Multiplex Nucleic Acid Amplification”, U.S. Pat. No. 5,882,856—“Universal Primer Sequence for Multiplex DNA Amplification”, U.S. Pat. No. 6,207,372 B1—“Universal Primer Sequence for Multiplex DNA Amplification”, and Lu, W. et. al. Nature Medicine Vol.5, pg 1081-1085 (1999), each incorporated herein by reference in their entirety. Standard PCR reaction conditions may be used, e.g., 1.5 mM MgCl₂, 50 mM KCl, 10 mM Tris-HCl, pH 8.3, 200 μM deoxynucleotide triphosphates (dNTPs), and 25-100 U/ml Taq polymerase (Perkin-Elmer, Norwalk, Conn.). Any commercially available thermal cycler may be used, such as, e.g., Perkin-Elmer 9600 cycler. One skilled in the art may vary the reaction components or conditions as appropriate, without departing from the spirit and scope of the present invention, for example, different thermostable polymerases may be used.

Suitable polymerases for amplification include, e.g., the DNA polymerases from Bacillus stearothermophilus, Thermus aquaticus, Pyrococcus furiosis, Thermococcus litoralis, and Thermus thermophilus, bacteriophage T4 and T7, and the E. coli DNA polymerase I Klenow fragment. Suitable RNA-directed DNA polymerases include, e.g., the reverse transcriptase from the Avian Myeloblastosis Virus, the reverse transcriptase from the Moloney Murine Leukemia Virus, and the reverse transcriptase from the Human Immunodeficiency Virus-I.

The concentration of each oligonucleotide in the reaction mixture can range from about 10 fM to about 10 μM. The optimal concentration for a first oligonucleotide, a first reverse oligonucleotide, and a second oligonucleotide is evaluated empirically, i.e., by performing individual amplification reactions using these oligonucleotides in varying concentrations. Generally, the second oligonucleotide is used in excess of the first and first reverse oligonucleotide, for example from about a 2-fold molar excess to about a 100-fold molar excess. In a currently preferred embodiment, the second oligonucleotide is used from about a 5 to about a 50-fold molar excess of the target specific first and first reverse oligonucleotides.

Where a second target sequence is to be simultaneously amplified, the initial conditions for amplification of the second target sequence are determined independently of the conditions for amplification of the first target sequence to confirm that target specific oligonucleotides to be included in a single multiplex amplification reaction require the same amplification conditions (i.e., temperature, duration of annealing and extension steps). For the multiplex reaction, the second universal oligonucleotide is used at a concentration from about a 2-fold molar excess to about a 100-fold molar excess over the total concentration of the target specific oligonucleotides.

For a multiplex amplification reaction of more than one target sequence, it is desirable to amplify the target sequences in roughly equal amounts. This may be accomplished by changing the concentration of the first and first reverse oligonucleotides in proportion to the third and third reverse oligonucleotides. Where three target sequences are to be amplified (a three-plex amplification), the proportion of the first and first reverse oligonucleotides, the third and third reverse oligonucleotides, and the fourth and fourth reverse oligonucleotides are adjusted. As described above, the second universal oligonucleotide is used at a concentration from about a 2-fold molar excess to about a 100-fold molar excess over the total concentration of the target specific oligonucleotides.

Finally, the reaction products are analyzed using any of several methods that are well-known in the art. Preferably, gel electrophoresis is used to rapidly resolve and identify each of the amplified target sequences. In a multiplex reaction, different amplified sequences are preferably of distinct sizes and thus can be resolved in a single gel. The gel matrix used will depend on the sizes of the target sequences. Alternatively, the amplification reaction mixture may treated with one or more enzymes prior to electrophoresis, for example, restriction endonucleases. Alternative methods of product analysis include without limitation dot-blot hybridization with allele-specific oligonucleotides, SSCP, sequencing, i.e., by hybridization or incorporation of fluorophores or dideoxynucleotides, or by extension reactions.

Amplification of a nucleic acid template as described above results in multiple copies of a template nucleic acid sequence. In one embodiment, a region of the sequence product is determined by annealing a sequencing primer to region of the template nucleic acid, and then contacting the sequencing primer with a DNA polymerase and a known nucleotide triphosphate, i.e., dATP, dCTP, dGTP, dTTP, or an analog of one of these nucleotides. The sequence primer can be any length or base composition, as long as it is capable of specifically annealing to a region of the amplified nucleic acid template. No particular structure is required for the sequencing primer is required so long as it is able to specifically prime a region on the amplified template nucleic acid. Preferably, the sequencing primer is complementary to a region of the template that is between the sequence to be characterized and the sequence hybridizable to the anchor primer. The sequencing primer is extended with the polymerase to form a sequence product. The extension is performed in the presence of one or more types of nucleotide triphosphates, and if desired, auxiliary binding proteins. Incorporation of the dNTP is determined by assaying for the presence of a sequencing byproduct. The nucleotide sequence of the sequencing product can also determined by measuring inorganic pyrophosphate (PPi) liberated from a nucleotide triphosphate (dNTP) as the NTP is incorporated into an extended sequence primer. This method of sequencing, termed Pyrosequencing TM. technology (PyroSequencing AB, Stockholm, Sweden) can be performed in solution (liquid phase) or as a solid phase technique. PPi-based sequencing methods are described generally in, e.g., WO9813523A1, Ronaghi, et al., 1996. Anal. Biochem. 242: 84-89, and Ronaghi, et al., 1998. Science 281: 363-365 (1998). These disclosures of PPi sequencing are incorporated herein in their entirety, by reference.

The invention further provides a reaction system for selectively detecting one or more target nucleic acid sequences in a population of nucleic acid molecules. The reaction system comprises a first oligonucleotide, a first reverse oligonucleotide, and a second oligonucleotide as described herein. Where it is desirable to amplify additional target sequences, a third and third reverse oligonucleotide, a fourth and fourth reverse oligonucleotide, a fifth and fifth reverse oligonucleotide etc., are also included. The reaction system may further comprise a polymerase such as one or more of the DNA polymerases from Bacillus stearothermophilus, Thermus aquaticus, Pyrococcus furiosis, Thermococcus litoralis, and Thermus thermophilus, bacteriophage T4 and T7, the E. coli DNA polymerase I Klenow fragment, the reverse transcriptase from the Avian Myeloblastosis Virus, the reverse transcriptase from the Moloney Murine Leukemia Virus, and the reverse transcriptase from the Human Immunodeficiency Virus-I or combinations of these enzymes.

The compositions of the present invention provide methods for selectively detecting one or more target nucleic acid sequences in a population of nucleic acid molecules. The target specific first and first reverse oligonucleotides and the second oligonucleotide are used to contact a population of nucleic acid molecules, then extended as described, thereby providing amplicons which can be detected by the techniques disclosed herein, and are indicative of one or more target sequences in the nucleic acid molecules. Such methods also provide for the detection of single nucleotide polymorphisms (SNP's) in a population of nucleic acid molecules.

The invention also provides for a kit comprising a plurality of oligonucleotides described herein, and optionally a polymerase. The oligonucleotides and polymerase are contained within one or more first sets of suitable packaging materials. The kit further comprises an instruction set for using the oligonucleotides and polymerase, and a second packaging material to contain the kit components.

Turning now to the figures, the invention is described in greater detail. In a standard amplification reaction employing more than a single pair of oligonucleotides, the obligatory imposition of a single-set of reaction conditions generally means that one of the oligonucleotide pairs will function more efficiently in priming, causing the target sequence specified by that oligonucleotide pair to be selectively amplified in the early cycles of amplification. Furthermore, the more efficient oligonucleotides will also be depleted from the reaction sooner than the less efficient ones, resulting in the increased accumulation of non-specific amplification products in later cycles of amplification. Clearly, these problems are magnified when it is desired to use multiple oligonucleotide pairs (>3-4) in a single reaction.

The compositions and methods of the present invention circumvent these problems by imposing a uniformly high degree of specificity on the annealing reactions that occur between different oligonucleotide pairs present in the mixture and their cognate target sequences in the nucleic acid template. During amplification, the target specific oligonucleotide pairs hybridize to the nucleic acid template, resulting in intermediate amplification products having the target specific sequences B and B′ at their 3′ termini, and a common sequence A at their 5′ termini where A comprises a sequence that does not hybridize to the target sequence. A second oligonucleotide adapter hybridizes to the resultant intermediate amplification products through the common sequence A, thus permitting subsequent rounds of amplification at high stringency and thereby lowering undesirable secondary amplification artifacts. This results in normalizing the degree of simultaneous amplification of different targets from a template nucleic acid in a single reaction.

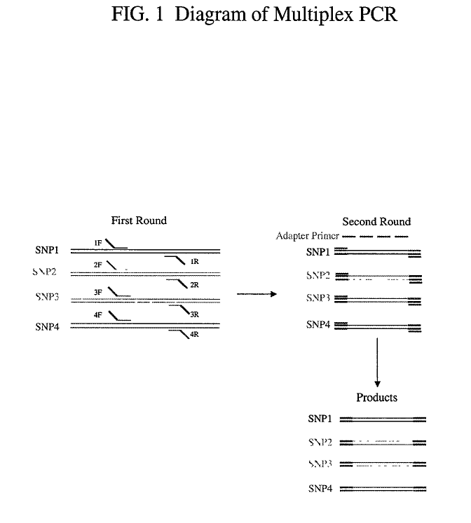

FIG. 1 illustrates a multiplex (four-plex) PCR according to the present invention. A nucleic acid template comprising the target sequences SNP1, SNP2, SNP3 and SNP4, is added to a reaction mixture comprising four pairs of target specific oligonucleotides (i.e., a first oligonucleotide and a first reverse oligonucleotide; a third oligonucleotide and a third reverse oligonucleotide; a fourth oligonucleotide and a fourth reverse oligonucleotide; and a fifth oligonucleotide and a fifth reverse oligonucleotide), and a second adapter oligonucleotide, along with standard PCR reaction components. The structure of the target specific oligonucleotides in this illustration are such that they all have a common sequence from the 5′ end, and a target specific sequence from the 3′ end, such that their extension products comprise the given target sequences (B and B′; C and C′; D and D′, and E and E′). The structure of the second adapter oligonucleotide is such that the nucleotide sequence is complementary to sequence A found at the 5′ end of the target specific oligonucleotides. The adaptor oligonucleotide is typically used in excess of the target specific oligonucleotides, for example, 1:10 or 1:50. Thus, during the first round of amplification, the eight low concentration target specific oligonucleotides will amplify the template nucleic acid to create four different double stranded amplicons. Each amplicon displays the common sequence A at its 5′ end. In subsequent rounds of amplification, the adapter oligonucleotide, added in excess, dominates the amplification reaction and anneals to all four amplicons in equal proportion, thereby permitting amplification of all four target SNP's in a single reaction.

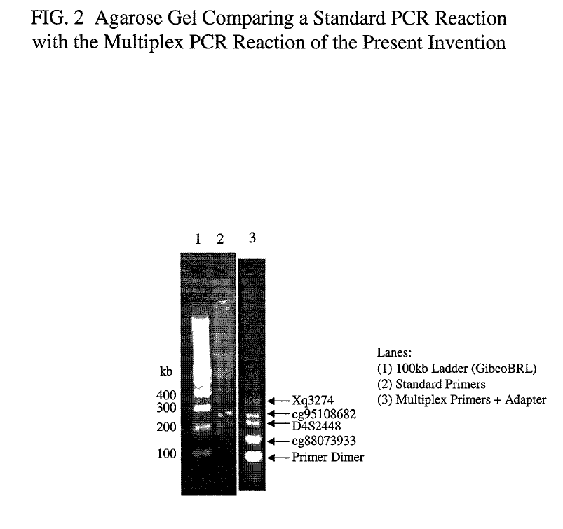

FIG. 2 illustrates amplification of target sequences, comparing a standard multiplex PCR reaction with the multiplex PCR of the present invention, to amplify four SNPs (cg88073933, D4S2448, cg95108682, and Xq3274) from one PCR mixture. Lane 1 illustrates the reference markers, 100 kb ladder (GibcoBRL). Lane 2 shows a standard multiplex PCR using eight standard 20mer oligonucleotides. Lane 3 illustrates a multiplex PCR of the present invention, using eight target specific 40mer oligonucleotides and one second adapter oligonucleotide. The resultant amplified products are resolved by electrophoresis on a 2.5% agarose gel. Lane 3 show amplification products of all four target sequences, the amplification products having sizes of 122 bp, 211 bp, 251 bp, and 327 bp respectively. Lane 2, by contrast, shows significantly lower levels of the two smallest amplification products.

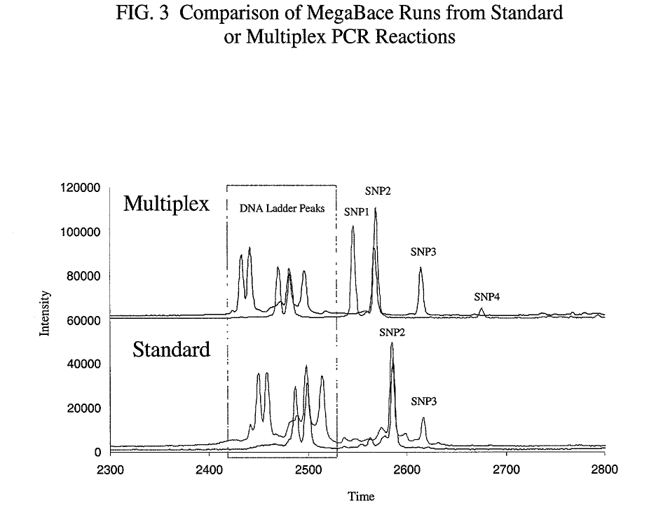

FIG. 3 illustrates MegaBACE runs of the amplification products from the PCR reactions shown in FIG. 2. After PCR amplification, the 10 μl reaction was taken through the remaining MegaBACE genotyping process by performing a SAP/ExoI digest step, a TDI extension, a second SAP digest, and Sephadex purification. Following purification, each reaction was run on the MegaBACE using standard genotyping protocols and running buffers. The results shown in FIG. 3 reveal that by using the multiplex PCR reaction of the present invention all four SNPs were correctly genotyped, while only two SNPs are seen in the standard multiplex PCR reaction.

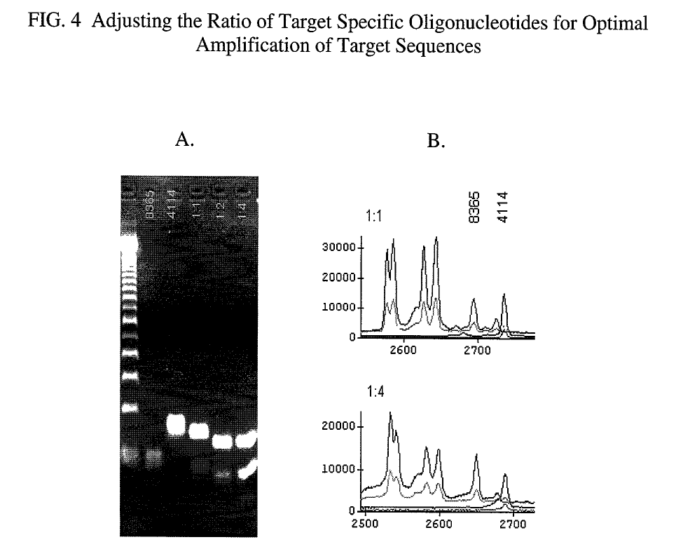

FIG. 4 illustrates PCR amplification reactions of a two-plexed sample, where the primer ratio between the target specific oligonucleotide pairs was adjusted to equalize the resultant products. Often one SNP pair had more PCR product by agarose gel than the other. Most of the time, the weaker band intensity was the smaller PCR product. Accordingly, the primer sets were adjusted empirically until relatively equal amplification product intensities were observed. Panel A illustrates an agarose gel used to resolve the reaction products from the PCR reactions. Lane one is a size reference standard, 100 kb ladder as described above. Lane two is a standard PCR amplification designed to amplify the 8365 SNP, and provides a positive control for the multiplex PCR. Lane three is a standard PCR amplification designed to amplify the 4114 SNP, again as a positive control. Target specific oligonucleotides for amplification of the two SNP's were used in a two-plex PCR in differing ratios. Lane four shows the results of a 1:1 ratio. Lane five shows the results of a 1:2 ratio and lane six shows the results of a 1:4 ratio. Panel B illustrates MegaBACE traces of the resulting amplification products shown as lanes three and five in Panel A. The results indicate that a oligonucleotide ratio of 1:4 between the target specific primers provided more balanced amplification of the two SNP's.

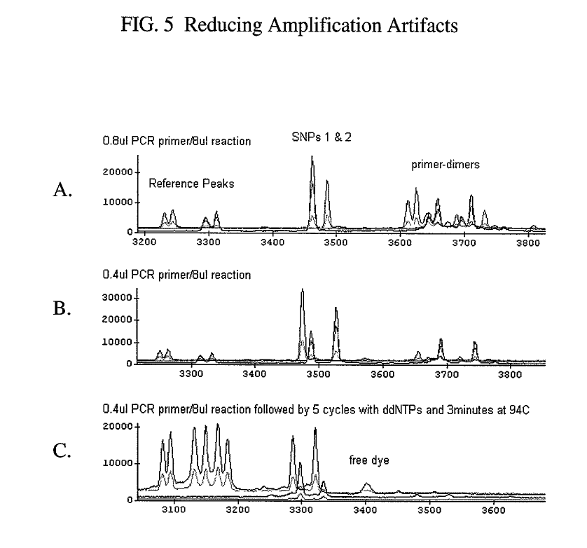

FIG. 5 illustrates the formation of primer dimers in an amplification reaction. Primer-dimers appeared consistently with one SNP pair (cg40367355 and cg88048627, shown as SNP's 1 and 2 respectively) but only sporadically with other SNP pairs. Panel A shows a MegaBACE trace of a two-plex amplification according to the invention. Numerous small amplification artifacts are visible. In Panel B, the total amount of oligonucleotides added to the reaction was halved, which reduced but did not eliminate the amplification artifacts. Panel C illustrates the two-plexed amplification using the reduced oligonucleotide concentrations shown in Panel B, and with the addition of chain terminating dideoxynucleotide triphosphates for five cycles after the two-plex PCR reaction, followed with a 94° C. denaturing step.

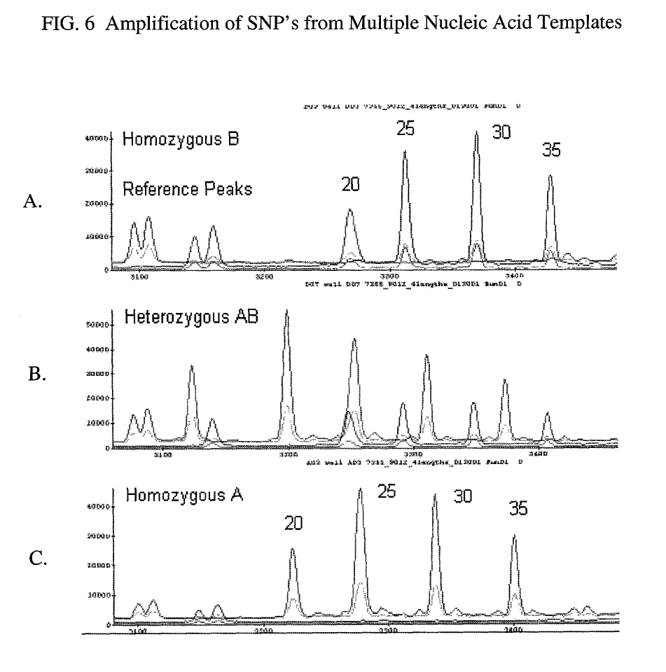

FIG. 6 illustrates MegaBACE traces of the resulting amplification products of polymorphic targets using various dye terminator combinations. SNPs were paired by their polymorphism similarity in order to reduce the quantities of dye terminators used, thereby reducing background peaks. SNPs that had opposite polymorphisms like C/T and A/G were paired together by using the forward primer for one and the reverse primer for the other to make their polymorphisms the same. Some dye terminator combinations migrated better than others, and this was also considered when choosing TDI oligonucleotide orientation. For example, BODIPY-fluorescein-U and BODIPY-TAMRA-C migrated right next to each other, but BODIPY-fluorescein-C and BODIPY-TAMRA-U and their A/G counterparts migrated much farther apart. Paired SNPs were extended with different length TDI oligonucleotides so that they could be distinguished from one another. The TDI oligonucleotide lengths were 20, 25, 30, and 35 bases, where t's were added to the 5′ end of a oligonucleotide until the appropriate length was reached. The separation between each length was almost equally spaced, and the allele products for each SNP were clearly distinguishable from those of adjacent SNPs.

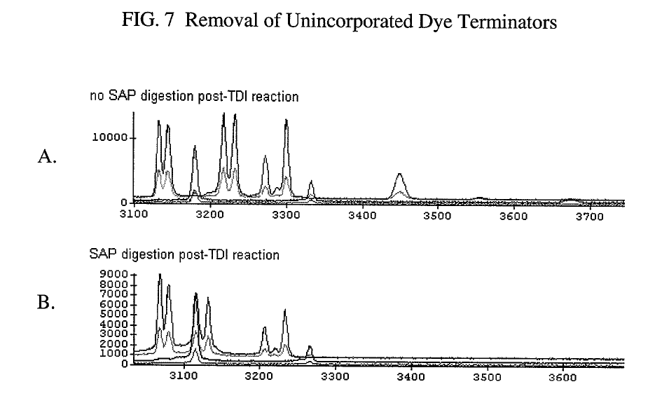

FIG. 7 illustrates traces of the amplification products before and following digestion of the unincorporated dye-conjugated free nucleotides with shrimp alkaline phosphatase (SAP). Sephadex purification did not remove the excess BODIPY-fluorescein (blue) dye terminators and removed most to all of the excess BODIPY-TAMRA (black) dye terminators. The free dye peaks seen were often large and varied in their distance from the allele peaks, depending on which SNP was amplified. For several SNPs a free dye peak overlapped with an allele peak, and for others they were seen several minutes later. Ultimately, adding a SAP digestion after the TDI reaction to degrade the free dyes completely removed them.

The following examples are intended to further illustrate the present invention without limiting the invention thereof.

### EXAMPLE 1

### Oligonucleotide Design

Sequence-specific oligonucleotides for the amplification of four target sequences in a single PCR were chosen without regard to hairpin formation and having a calculated ΔG for duplexing below −10 kcal/mole (see, Table 1). The Tₘ of these oligonucleotides was determined by the A+T/G+C method. To evaluate potential dimer formations oligonucleotides were analyzed using Amplify 1.2 software (University of Wisconsin, Department of Genetics, Madison, Wis.). Oligonucleotides were synthesized by standard phosphoramidite reactions, then were HPLC purified and quantitated by spectrophotometry.

TABLE 1

Oligonucleotides for a Four-Plex PCR

| | | | |
|---|---|---|---|
| | | | SEQ |
| Target | | | ID |
| SNP | NAME | Sequence 5′-3′ | NO. |

| | | | |
|---|---|---|---|
| CG88073933 | 3933 | GCGGTCCCAAAAGGGTCAGTCCCA | 1 |

| | |
|---|---|
| Forward | AATGCCAAGGGGGAAAACC |

| | | | |
|---|---|---|---|
| CG88073933 | 3933 | GCGGTCCCAAAAGGGTCAGTAGGA | 2 |

| | |
|---|---|
| Reverse | TGGTCTTGGCAGAGCCACCA |

| | | | |
|---|---|---|---|
| CG95108682 | 8682 | GCGGTCCCAAAAGGGTCAGTTGGC | 3 |

| | |
|---|---|
| Forward | AGGAGGCTCTGGGCTCAT |

| | | | |
|---|---|---|---|
| CG95108682 | 8682 | GCGGTCCCAAAAGGGTCAGTCACT | 4 |

| | |
|---|---|
| Reverse | GCCTGGCTTCCGGACCAT |

| | | | |
|---|---|---|---|
| D4S2448 | D4S2448 | GCGGTCCCAAAAGGGTCAGTCGCC | 5 |

| | |
|---|---|
| Forward | ATGCTGCAGTGACCACA |

| | | | |
|---|---|---|---|
| D4S2448 | D4S2448 | GCGGTCCCAAAAGGGTCAGTCAGA | 6 |

| | |
|---|---|
| Reverse | AGGCATGGCTCTTCTCCATGA |

| | | | |
|---|---|---|---|
| Xq3274 | Xq3274 | GCGGTCCCAAAAGGGTCAGTTTGG | 7 |

| | |
|---|---|
| Forward | GAGGCCGAGGCAGACG |

| | | | |
|---|---|---|---|
| Xq3274 | Xq3274 | GCGGTCCCAAAAGGGTCAGTTGAA | 8 |

| | |
|---|---|
| Reverse | TGCGAACTCCTGGGCTCAA |

| | | | |
|---|---|---|---|
| N/A | adapter | GCGGTCCCAAAAGGGTCAGT | 9 |

### EXAMPLE 2

### Preparation of Template Nucleic Acids

Whole blood samples were collected in high glucose ACD Vacutainers® (Beckton Dickenson & Co., Franklin Lanes, N.J.). Following centrifugation, the buffy coat was collected and lysed with two washes of a 10:1 (v/v) solution of 14 mM NH₄Cl and 1 mM NaHCO₃. The lymphocytes were harvested by centrifugation, resuspended in lysis buffer (10 mM Tris-HCl, pH 8.0, 0.4 M NaCl, 2 mM EDTA, 0.5% SDS, 500 μg/ml proteinase K) and incubated overnight at 37° C. Samples were then extracted with ¼th volume of saturated NaCl, and the DNA was collected by ethanol precipitation. The final DNA pellet was washed with 70% ethanol, air dried and dissolved in TE (10 mM Tris-HCl, pH 7.5, 1 mM EDTA).

Buccal cell samples were obtained by brushing the lining of the buccal cavity for 30 seconds with a sterile cytology brush (Scientific Products #S7766-1a). DNA was prepared by immersing the brushes in 600 μl of 50 mM NaOH in 1.2 ml 96-well polypropylene tubes (USA/Scientific Plastics, Ocala, Fla.) and vortexed. The tubes, still containing the brushes, were heated to 95° C. for 5 min. and the brushes were carefully removed. The lysates were neutralized with 60 μl of 1 M Tris-HCl (pH 8.0) and vortexed. Samples were stored at 4° C.

### EXAMPLE 3

### Amplification Reactions

Amplification of target sequences were performed in a 50 μl final reaction volume using 20 μl (1 μg) of genomic DNA prepared as described above. In, addition, the following reagents were used:

20 μl (2.5 μg total weight) of the oligonucleotide primers listed in Example 1, 5 μl of 10× Eppendorf Taq Buffer (500 mM KCl, 100 mM Tris-HCl (pH 8.3), 15 mM MgCl2, 1% Triton X-100), 4 μl (2.5 mM) dNTP's, 0.5 μl (5 units/μl) Eppendorf Taq Polymerase, and 0.5 μl water.

Amplifications were carried out using a Perkin-Elmer 9600 themocycler (Perkin-Elmer, Norwalk, Conn.) as follows:

Step 1-95° C.-3 min

Step 2-95° C.-3 s
Step 3-78° C.-5 s
Step 4-ramp down 0.1° C./s to 68° C.
Step 5-68° C.-90 s
Step 6-repeat steps 2-5 10 times
Step 7-95° C.-30 s
Step 8-65° C.-90 s
Step 9-repeat steps 8-9 23 times
Step 10-72° C.-5 min
Step 11-4° C.-hold (storage conditions)

A control multiplex PCR amplification was performed in an identical manner, but the adapter primer (SEQ ID NO. 9) was omitted from the reaction mixture.

After completion of the reaction, a sample of the reaction products were loaded directly onto a 2.5% agarose gel and subjected to electrophoresis, then the gel was subjected to ethidium bromide-staining. The amplification products were visualized with a UV transilluminator and photographed with an Alpha Innotech IS-500 Digital Imaging System version 1.97 (Sun BIO Science, Inc., Branford, Conn.). The results are shown as FIG. 2. The multiplex PCR of the present invention, utilizing the adapter primer (SEQ ID NO.:9) amplified all four SNP's while the control reaction amplified only two SNP's. In addition, the band intensities of the multiplex PCR of the present invention suggest a much higher concentration of amplification products.

Several experiments were run to determine if SNPs could be paired by iCycler® (BioRad) threshold cycles. The purpose was to find a way to pair SNPs that would be more accurate than pairing them by agarose gel band intensity and would require little human interpretation. Correlations were found between threshold cycle and band intensity, and were fairly consistent (Table 1 and notebook 575 pages 25 and 78). After learning that band intensity does not significantly affect peak intensity, no further experiments were done with the iCycler®. In addition, PCR products were checked on an agarose gel for primer QC, so their intensities were relatively recorded in relation to other bands on the gel.

TABLE 2
iCycler ® Threshold Cycles

| | Agarose Gel Product | | Threshold Cycle | |
|---|---|---|---|---|
| SNP | size | intensity | average | range |
| cg9076 | 204 | lots of product | 11.6 | 11.6-16.8 |
| cg4114 | 175 | | 11.9 | |
| 8368 | 226 | | 12.0 | |
| 8366 | 156 | | 12.2 | |
| cg3933 | ˜200 | | 12.3 | |
| 9532 | 250 | | 13.1 | |
| cg5825 | ˜200 | | 13.9 | |
| cg0091 | ˜175 | | 16.2 | |
| 1021 | 168 | | 16.8 | |
| cg8648 | 101 | some product | 10.1 | 10.1-15.4 |
| 8365 | 101 | | 13.5 | |
| cg9360 | 133 | | 15.4 | |
| cg3665 | 251 | little product | 18.1 | 18.1 |
| 1661 | 211 | (incorrect band size) | 16.6 | 16.6-17.1 |
| 8369 | 313 | (incorrect band size) | 17.1 | |
| 8367 | 211 | no product | 21.8 | 21.8-25.9 |
| 2360 | 277 | | 22.1 | |
| 1071 | 312 | | 22.6 | |
| 2K12 | 203 | | 23.2 | |
| cg2878 | 175 | | 24.0 | |
| cg1974 | 238 | | 23.9 | |
| cg8747 | 134 | | 23.9 | |
| 1415 | 273 | | 25.8 | |
| Ecad | 204 | | 25.9 | |

Often one SNP pair had more PCR product by agarose gel than the other. Most of the time, the weaker band intensity was seen as the smaller PCR product. The ratio of SNP specific oligonucleotide pairs was thus adjusted to equalize the product intensities. As shown in FIG. 4, 1:4 was found to be a useful equalization ratio, i.e., the first oligonucleotide and first reverse oligonucleotide specific to the target subsequence B and B′ contained within the SNP 8365 was used in four-fold excess of the third oligonucleotide and third reverse oligonucleotide specific for the target subsequence C and C′ contained within the SNP 4114. The second adaptor primer having the universal sequence A was used in 10-fold excess of the total molar concentration of the target specific primers as equalized.

The amplification products were used in subsequent template directed dye terminator incorporation reactions (TDI) for MegaBACE genotyping runs (discussed in Example Three below), and the results are shown in FIG. 3. Again, the multiplex PCR of the present invention amplified all four SNP's, while the control reaction without the adapter primer shows only two amplification products.

EXAMPLE THREE

### MegaBACE Genotyping

Following the multiplex PCR, the amplification reaction was subjected MegaBACE genotyping chemistry. Briefly, this consisted of an SAP/ExoI digestion step, TDI extension, SAP digestion, and gel purification of the final reaction product.

The shrimp alkaline phosphatase (SAP) and Exonuclease I digestion was performed as follows: A reaction mixture consisting of 10 μl of the amplification mixture, 6.32 μl water, 0.8 μl of 10× SAP buffer (0.5M Tris-HCl, 50 mM MgCl₂ pH 8.5), 0.8 μl of 1U/ul SAP (Roche), and 0.08 μl of 10 U/ul Exonuclease I (USB Corporation) was incubated for 1 hour at 37° C. and the enzyme was inactivated by heating to 95° C. for 15 minutes. The mixture was stored at 4° C. prior to the TDI reaction.

Dye terminators were incorporated into the amplification products as described in Chen, et al., Genome Research 9: 492-498 1999); Chen, et al., Nucleic Acids Research 25(2): 347-353 (1997), U.S. Pat. No. 6,180,408: and U.S. Pat. No. 6,355,433, each incorporated herein by reference. Sequences for the oligonucleotide primers used in the dye terminator reaction are given in Table 3.

TABLE 3

Dye Terminator Incorporation Oligonucleotides

| | | |
|---|---|---|
| | | SEQ |
| | | ID |
| Name | Sequence 5′-3′ | NO. |

| | | |
|---|---|---|
| cg95108682F21 | CCTGACCACAGGCTCTTGAAG | 10 |
| Xq3274R25 | TTTTTTTCGAACTCCTGGGCTCAAG | 11 |
| cg88073933R30 | TTTTTTTGATGTCTTTAGTATGCAGCTCAA | 12 |
| D4S2448F35 | TTTTTTTTTTTTTTTTGCCCAACTCAAAGACTGG | 13 |

A

Incorporation of dye-terminators was accomplished using the following reaction mixture:

3.168 μl water, 1.6 μl 5× reaction buffer (0.3M Tris base, 0.25M KCl, 25 mM NaCl, 12 mM MgCl₂, 40% glycerol, pH 9.0), 0.8 μl (10 μM final concentration) of each oligonucleotide cg95108682F21, Xq3274R25, cg88073933R30, and D4S2448F35, 0.008 μl 100 μM final concentration Dye Terminator 1 (NEN; Perkin Elmer), 0.008 μl 100 μM final concentration Dye Terminator 2, and 0.016 μl 32U/μl ThermoSequenase (Amersham)

The dye-terminator incorporation cycling conditions used were:

| | |
|---|---|
| Step 1: | 95° C. for 2 minutes |
| Step 2 through Step 16: | 95° C. 20 seconds, 70° C. 5 seconds, 55° C. 30 |
| | seconds |
| Step 17: | 4° C. hold (storage conditions). |

Following the TDI extension, a second SAP digestion was performed to degrade unincorporated dye conjugated nucleotides. The reaction conditions were: 6.4 μl water, 0.8 μl 10× SAP buffer, 0.8 μl 1U/ul SAP, incubated for 1 hour at 37° C., then stored at 4° C. prior to MegaBACE runs. The results improved the resolution of the MegaBACE runs as shown in FIG. 7.

Additional modifications were investigated to further reduce the formation of primer dimers and improve the resolution. As shown in FIG. 5, a twofold reduction of the total oligonucleotide concentration dramatically reduced the formation of primer dimers. Likewise, the addition of about 0.1 to about 100 nM ddNTP's for five cycles after the PCR amplification reaction followed by a three minute denaturing step at 94 degrees also reduced the formation or appearance of primer dimers (see, FIG. 5). In addition, SNPs were paired by their polymorphism similarity to reduce background peaks. SNPs that had opposite polymorphisms like C/T and A/G were paired together by using the forward oligonucleotide for one and the reverse oligonucleotide for the other to make their polymorphisms the same. Some dye terminator combinations migrated better than others, and this was also considered when choosing TDI oligonucleotide orientation. For example, BODIPY-fluorescein-U and BODIPY-TAMRA-C migrated right next to each other, but BODIPY-fluorescein-C and BODIPY-TAMRA-U and their A/G counterparts migrated much farther apart (see, FIG. 6).

### Equivalents

From the foregoing detailed description of the specific embodiments of the invention, it should be apparent that unique compositions and methods of performing multiplex amplification of target sequences has been described. Although particular embodiments have been disclosed herein in detail, this has been done by way of example for purposes of illustration only, and is not intended to be limiting with respect to the scope of the appended claims that follow. In particular, it is contemplated by the inventor that substitutions, alterations, and modifications may be made to the invention without departing from the spirit and scope of the invention as defined by the claims. For instance, the choice of dye terminators or oligonucleotide sequences is believed to be matter of routine for a person of ordinary skill in the art with knowledge of the embodiments described herein.

We claim:

1. A plurality of oligonucleotides, the plurality comprising
(a) a first oligonucleotide having a 5′ terminus and a 3′ terminus and comprising an A subsequence and a B subsequence, wherein said B subsequence is provided at the 3′ terminus of said first oligonucleotide, and wherein the B subsequence hybridizes to a target nucleic acid sequence and the A subsequence does not hybridize to said target nucleic acid sequence; and
(b) a second oligonucleotide having a 5′ terminus and a 3′ terminus, the second oligo nucleotide comprising said A subsequence at its 3′ terminus.

2. The plurality of claim 1, further comprising a first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to a target nucleic acid sequence and can be extended to form an extension product comprising a sequence complementary to said B subsequence, and the A subsequence does not hybridize to said target nucleic acid sequence.

3. The plurality of claim 1, wherein the first oligonucleotide includes said A subsequence at its 5′ terminus.

4. The plurality of claim 1, wherein the second oligonucleotide includes said A subsequence at its 5′ terminus.

5. The plurality of claim 1, wherein the second oligonucleotide comprises the nucleic acid sequence of SEQ ID NO: 9.

6. The plurality of claim 1, wherein the B subsequence comprises the nucleic acid sequence selected from the group consisting of SEQ ID NO: 1, 3, 5 and 7 and the B¹ subsequence comprises the nucleic acid sequence selected from the group consisting of SEQ ID NO: 2, 4, 6 and 8.

7. The plurality of claim 1, wherein the second oligonucleotide is present at a higher concentration in said plurality than said first oligonucleotide.

8. The plurality of claim 1, wherein the second oligonucleotide is present at 2, 5, 10, 15, 20, 50-fold higher molar concentration than said first oligonucleotide.

9. The plurality of claim 1, wherein the first oligonucleotide is 30-50 nucleotides in length.

10. The plurality of claim 1, wherein the first oligonucleotide is 35-45 nucleotides in length.

11. The plurality of claim 1, wherein the first oligonucleotide is about 40 nucleotides in length.

12. The plurality of claim 1, wherein the second oligonucleotide is 15-25 nucleotides in length.

13. The plurality of claim 1, wherein the second oligonucleotide is 17-23 nucleotides in length.

14. The plurality of claim 1, wherein the third oligonucleotide is 20 nucleotides in length.

15. The plurality of claim 1, further comprising a third oligonucleotide having a 5′ terminus and a 3′ terminus, the third oligonucleotide comprising said A subsequence at its 5′ terminus and a C subsequence at its 3′ terminus, wherein the C subsequence hybridizes to a target nucleic acid sequence and the A subsequence does not hybridize to said target nucleic acid sequence.

16. The plurality of claim 1, further comprising a third reverse oligonucleotide having a 5′ terminus and a 3′ terminus and comprising an A subsequence and a C′ subsequence, wherein said B′ subsequence is provided at the 3′ terminus of said oligonucleotide, and wherein the C′ subsequence hybridizes to a target nucleic acid sequence and can be extended to form an extension product comprising a sequence complementary to said C subsequence, and the A subsequence does not hybridize to said target nucleic acid sequence.

17. The plurality of claim 1, further comprising a fourth oligonucleotide having a 5′ terminus and a 3′ terminus, the fourth oligonucleotide comprising said A subsequence at its 5′ terminus and a D subsequence at its 3′ terminus, wherein the D subsequence hybridizes to a target nucleic acid sequence and the A subsequence does not hybridize to said target nucleic acid sequence.

18. The plurality of claim 1, further comprising a fourth reverse oligonucleotide having a 5′ terminus and a 3′ terminus and comprising an A subsequence and a D′ subsequence, wherein said B′ subsequence is provided at the 3′ terminus of said oligonucleotide, and wherein the D′ subsequence hybridizes to a target nucleic acid sequence and can be extended to form an extension product comprising a sequence complementary to said D subsequence, and the A subsequence does not hybridize to said target nucleic acid sequence.

19. The plurality of claim 1, further comprising a fifth oligonucleotide having a 5′ terminus and a 3′ terminus, the fifth oligonucleotide comprising said A subsequence at its 5′ terminus and an E subsequence at its 3′ terminus, wherein the E subsequence hybridizes to a target nucleic acid sequence and the A subsequence does not hybridize to said target nucleic acid sequence.

20. The plurality of claim 1, further comprising a fifth reverse oligonucleotide having a 5′ terminus and a 3′ terminus and comprising an A subsequence and an E′ subsequence, wherein said B subsequence is provided at the 3′ terminus of said oligonucleotide, and wherein the E′ subsequence hybridizes to a target nucleic acid sequence and can be extended to form an extension product comprising a sequence complementary to said E subsequence, and the A subsequence does not hybridize to said target nucleic acid sequence.

21. A reaction system for selectively detecting one or more target nucleic acid sequences in a population of nucleic acid molecules, the reaction system comprising
(a) a population of starting nucleic acid molecules known to or suspected of containing at least one target nucleic acid sequence;
(b) a first oligonucleotide having a 5′ terminus and a 3′ terminus and comprising an A subsequence and a B subsequence, wherein said B subsequence is provided at the 3′ terminus of said oligonucleotide, and wherein the B subsequence hybridizes to said at least one target nucleic acid sequence and the A subsequence does not hybridize to said at least one target nucleic acid sequence; and
(c) a second oligonucleotide having a 5′ terminus and a 3′ terminus, the second nucleotide comprising said A subsequence at its 3′ terminus.

22. The reaction system of claim 21, further comprising a first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to said at least one target nucleic acid sequence and can be extended to form an extension product comprising a sequence complementary to said B subsequence, and the A subsequence does not hybridize to said target nucleic acid sequence.

23. The reaction system of claim 21, wherein the first oligonucleotide includes said A subsequence at its 5′ terminus.

24. The reaction system of claim 21, wherein the second oligonucleotide includes said A subsequence at its 5′ terminus.

25. The reaction system of claim 21, wherein the second oligonucleotide is present at a higher concentration in said plurality than said first oligonucleotide.

26. The reaction system of claim 21, wherein the second oligonucleotide is present at 2, 5, 10, 15, 20, 50-fold higher molar concentration than said first oligonucleotide.

27. The reaction system of claim 21, wherein the first oligonucleotide is 30-50 nucleotides in length.

28. The reaction system of claim 21, wherein the first oligonucleotide is 35-45 nucleotides in length.

29. The reaction system of claim 21, wherein the first oligonucleotide is about 40 nucleotides in length.

30. The reaction system of claim 21, wherein the second oligonucleotide is 15-25 nucleotides in length.

31. The reaction system of claim 21, wherein the second oligonucleotide is 17-23 nucleotides in length.

32. The reaction system of claim 21, wherein the third oligonucleotide is 20 nucleotides in length.

33. The reaction system of claim 21, comprising a third oligonucleotide having a 5′ terminus and a 3′ terminus, the third oligonucleotide comprising said A subsequence at its 5′ terminus and a C subsequence at its 3′ terminus, wherein the C subsequence hybridizes to a target nucleic acid sequence and the A subsequence does not hybridize to said target nucleic acid sequence.

34. The reaction system of claim 21, further comprising a third reverse oligonucleotide having a 5′ terminus and a 3′ terminus and comprising an A subsequence and a C′ subsequence, wherein said B′ subsequence is provided at the 3′ terminus of said oligonucleotide, and wherein the C′ subsequence hybridizes to a target nucleic acid sequence and can be extended to form an extension product comprising a sequence complementary to said C subsequence, and the A subsequence does not hybridize to said target nucleic acid sequence.

35. The reaction system of claim 21, further comprising a fourth oligonucleotide having a 5′ terminus and a 3′ terminus, the fourth oligonucleotide comprising said A subsequence at its 5′ terminus and a D subsequence at its 3′ terminus, wherein the D subsequence hybridizes to a target nucleic acid sequence and the A subsequence does not hybridize to said target nucleic acid sequence.

36. The reaction system of claim 21, further comprising a fourth reverse oligonucleotide having a 5′ terminus and a 3′ terminus and comprising an A subsequence and a D′ subsequence, wherein said B′ subsequence is provided at the 3′ terminus of said oligonucleotide, and wherein the D′ subsequence hybridizes to a target nucleic acid sequence and can be extended to form an extension product comprising a sequence complementary to said D subsequence, and the A subsequence does not hybridize to said target nucleic acid sequence.

37. The reaction system of claim 21, further comprising a fifth oligonucleotide having a 5′ terminus and a 3′ terminus, the fifth oligonucleotide comprising said A subsequence at its 5′ terminus and an E subsequence at its 3′ terminus, wherein the E subsequence hybridizes to a target nucleic acid sequence and the A subsequence does not hybridize to said target nucleic acid sequence.

38. The reaction system of claim 21, further comprising a fifth reverse oligonucleotide having a 5′ terminus and a 3′ terminus and comprising an A subsequence and an E′ subsequence, wherein said B subsequence is provided at the 3′ terminus of said oligonucleotide, and wherein the E′ subsequence hybridizes to a target nucleic acid sequence and can be extended to form an extension product comprising a sequence complementary to said E subsequence, and the A subsequence does not hybridize to said target nucleic acid sequence.

39. The reaction system of claim 21, further comprising a polymerase.

40. The reaction system of claim 39, wherein the polymerase is selected from the group consisting of {favored polymerase)

41. A kit for selectively detecting one or more target nucleic acid sequences in a population of nucleic acid molecules, the kit comprising
(a) a first oligonucleotide having a 5′ terminus and a 3′ terminus and comprising an A subsequence and a B subsequence, wherein said B subsequence is provided at the 3′ terminus of said oligonucleotide, and wherein the B subsequence hybridizes to a target nucleic acid sequence and the A subsequence does not hybridize to said target nucleic acid sequence; and
(b) a second oligonucleotide having a 5′ terminus and a 3′ terminus, the second nucleotide comprising said A subsequence at its 3′ terminus.

42. The kit of claim 41, further comprising a first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to a target nucleic acid sequence and can be extended to form an extension product comprising a sequence complementary to said B subsequence, and the A subsequence does not hybridize to said target nucleic acid sequence.

43. The kit of claim 41, further comprising instructions for the kit.

44. The kit of claim 41, wherein the first oligonucleotide includes said A subsequence at its 5′ terminus.

45. The kit of claim 41, wherein the second oligonucleotide includes said A subsequence at its 5′ terminus

46. The kit of claim 41, wherein the second oligonucleotide is present at a higher concentration than said first oligonucleotide.

47. The kit of claim 41, wherein the second oligonucleotide is present at 2, 5, 10, 15, 20, 50-fold higher molar concentration than said first oligonucleotide.

48. The kit of claim 41, wherein the first oligonucleotide is 30-50 nucleotides in length.

49. The kit of claim 41, wherein the first oligonucleotide is 35-45 nucleotides in length.

50. The kit of claim 41, wherein the first oligonucleotide is about 40 nucleotides in length.

51. The kit of claim 41, wherein the second oligonucleotide is 15-25 nucleotides in length.

52. The kit of claim 41, wherein the second oligonucleotide is 17-23 nucleotides in length.

53. The kit of claim 41, wherein the third oligonucleotide is 20 nucleotides in length.

54. The kit of claim 41, further comprising a third oligonucleotide having a 5′ terminus and a 3′ terminus, the third oligonucleotide comprising said A subsequence at its 5′ terminus and a C subsequence at its 3′ terminus, wherein the C subsequence hybridizes to a target nucleic acid sequence and the A subsequence does not hybridize to said target nucleic acid sequence.

55. The kit of claim 41, further comprising a third reverse oligonucleotide having a 5′ terminus and a 3′ terminus and comprising an A subsequence and a C′ subsequence, wherein said B′ subsequence is provided at the 3′ terminus of said oligonucleotide, and wherein the C′ subsequence hybridizes to a target nucleic acid sequence and can be extended to form an extension product comprising a sequence complementary to said C subsequence, and the A subsequence does not hybridize to said target nucleic acid sequence.

56. The kit of claim 41, further comprising a fourth oligonucleotide having a 5′ terminus and a 3′ terminus, the fourth oligonucleotide comprising said A subsequence at its 5′ terminus and a D subsequence at its 3′ terminus, wherein the D subsequence hybridizes to a target nucleic acid sequence and the A subsequence does not hybridize to said target nucleic acid sequence.

57. The kit of claim 56, further comprising a fourth reverse oligonucleotide having a 5′ terminus and a 3′ terminus and comprising an A subsequence and a D′ subsequence, wherein said B′ subsequence is provided at the 3′ terminus of said oligonucleotide, and wherein the D′ subsequence hybridizes to a target nucleic acid sequence and can be extended to form an extension product comprising a sequence complementary to said D subsequence, and the A subsequence does not hybridize to said target nucleic acid sequence.

58. The kit of claim 56, further comprising a fifth oligonucleotide having a 5′ terminus and a 3′ terminus, the third oligonucleotide comprising said A subsequence at its 5′ terminus and an E subsequence at its 3′ terminus, wherein the E subsequence hybridizes to a target nucleic acid sequence and the A subsequence does not hybridize to said target nucleic acid sequence.

59. The kit of claim 58, further comprising a fourth reverse oligonucleotide having a 5′ terminus and a 3′ terminus and comprising an A subsequence and an E′ subsequence, wherein said B subsequence is provided at the 3′ terminus of said oligonucleotide, and wherein the E′ subsequence hybridizes to a target nucleic acid sequence and can be extended to form an extension product comprising a sequence complementary to said E subsequence, and the A subsequence does not hybridize to said target nucleic acid sequence.

60. A method for selectively detecting one or more target nucleic acid sequences in a population of nucleic acid molecules, the method comprising
(a) contacting a population of starting nucleic acid molecules known to or suspected of containing at least one target nucleic acid sequence with an effective amount of a first oligonucleotide and an effective amount of a first reverse oligonucleotide to form a primed first oligonucleotide complex and a primed first reverse oligonucleotide complex,
said first oligonucleotide having a 5′ terminus and a 3′ terminus and comprising an A subsequence and a B subsequence, wherein said B subsequence is provided at the 3′ terminus of said oligonucleotide, and wherein the B subsequence hybridizes to said target nucleic acid sequence and the A subsequence does not hybridize to said target nucleic acid sequence, thereby forming an annealed first oligonucleotide-target nucleotide complex;
said first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to a target nucleic acid sequence and can be extended to form an extension product comprising a sequence complementary to said B subsequence, and the A subsequence does not hybridize to said target nucleic acid sequence, thereby forming an annealed first reverse oligonucleotide-target nucleotide complex;
(b) extending said annealed first oligonucleotide complex and annealed first reverse oligonucleotide complex with a polymerase to form a first extended oligonucleotide sequence and first reverse extended oligonucleotide sequence;
(c) combining said first extended oligonucleotide sequence and first reverse extended oligonucleotide sequence with an effective amount of a second oligonucleotide to form annealed second oligonucleotide complex, wherein said second oligonucleotide has a 5′ terminus and a 3′ terminus, the second nucleotide comprising said A subsequence at its 3′ terminus;
(d) extending said annealed second oligonucleotide-target nucleotide complex and annealed first reverse oligonucleotide-target nucleotide complex with a polymerase, thereby forming extended second oligonucleotide sequences; and
(e) detecting said extended second oligonucleotide sequences, thereby selectively amplifying one or more target nucleic acid sequences in a population of nucleic acid molecules.

61. The method of claim 60, further comprising contacting said starting population of nucleic acid molecules with said second oligonucleotide.

62. The method of claim 61, wherein said second oligonucleotide is present at a higher concentration than said first oligonucleotide.

63. A method for detecting a single nucleotide polymorphism in a population of nucleic acid molecules, the method comprising
(a) contacting a population of starting nucleic acid molecules known to or suspected of containing at least one polymorphic nucleic acid sequence with an effective amount of a first oligonucleotide and an effective amount of a first reverse oligonucleotide to form a primed first oligonucleotide complex and a primed first reverse oligonucleotide complex,
said first oligonucleotide having a 5′ terminus and a 3′ terminus and comprising an A subsequence and a B subsequence, wherein said B subsequence is provided at the 3′ terminus of said oligonucleotide, and wherein the B subsequence hybridizes to said polymorphic nucleic acid sequence and the A subsequence does not hybridize to said polymorphic nucleic acid sequence, thereby forming an annealed first oligonucleotide-polymorphic nucleotide complex;
said first reverse oligonucleotide having a 5′ terminus and a 3′ terminus and comprising an A subsequence and a B′ subsequence, wherein said B′ subsequence is provided at the 3′ terminus of said oligonucleotide, and wherein the B′ subsequence hybridizes to a polymorphic nucleic acid sequence and can be extended to form an extension product comprising a sequence complementary to said B subsequence, and the A subsequence does not hybridize to said polymorphic nucleic acid sequence, thereby forming an annealed first reverse oligonucleotide-target nucleotide complex;
(b) extending said annealed first oligonucleotide complex and annealed first reverse oligonucleotide complex with a polymerase to form a first extended oligonucleotide sequence and first reverse extended oligonucleotide sequence;
(c) combining said first extended oligonucleotide sequence and first reverse extended oligonucleotide sequence with an effective amount of a second oligonucleotide to form annealed second oligonucleotide complex, wherein said second oligonucleotide has a 5′ terminus and a 3′ terminus, the second nucleotide comprising said A subsequence at its 3′ terminus;
(d) extending said annealed second oligonucleotide-polymorphic nucleotide complex and annealed first reverse oligonucleotide-target nucleotide complex with a polymerase, thereby forming extended second oligonucleotide sequences; and
(e) detecting said extended second oligonucleotide sequences, thereby identifying a single nucleotide polymorphic sequence in a population of nucleic acid molecules.

64. The method of claim 63, further comprising contacting said starting population of nucleic acid molecules with said second oligonucleotide.

65. The method of claim 63, wherein said second oligonucleotide is present at a higher concentration than said first oligonucleotide.
